(19) United States
(12) Patent Application Publication
(10) Pub. No.: US 2002/0132318 A1
(43) Pub. Date: Sep. 19, 2002

(54) Fluorescent proteins

(76) Inventors: Ming Zhao, San Diego, CA (US); Mingxu Xu, La Jolla, CA (US); Ping Jiang, San Diego, CA (US); Meng Yang, San Diego, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/060,857

(22) Filed: Jan. 29, 2002

Related U.S. Application Data
(60) Provisional application No. 60/264,932, filed on Jan. 29, 2001.

(51) Int. Cl. C12N 9/00; C07H 21/04; C07K 14/435; C12N 5/06; C12P 21/02

(52) U.S. Cl. 435/183; 435/69.1; 435/320.1; 435/325; 530/350; 536/23.2

(57) ABSTRACT

Improved forms of fluorescent protein with high fluorescence and low toxicity are disclosed.

### CROSS-REFERENCE TO RELATED APPLICATIONS

This application claims priority under 35 U.S.C. § 119(e) from provisional application 60/264,932 filed Jan. 29, 2001. The contents of this application are incorporated herein by reference.

### TECHNICAL FIELD

The invention relates to new forms of “green fluorescent protein” and their uses. Specifically, the invention is directed to a particular GFP and variants thereof which are bright and nontoxic.

### BACKGROUND ART

Green fluorescent protein (GFP) was initially isolated from Aequorea victoria by Chalfie, U.S. Pat. No. 5,491,084. Modifications were made to the amino acid sequence to enhance brightness as reported by Ward and Chalfie in PCT publication WO 95/21191. Tsien, as disclosed in U.S. Pat. Nos. 5,625,048 and 5,777,079, provided modified forms of this protein which exhibited differing spectral characteristics and provided fluorescence of various colors. In addition, modifications were made to the nucleotide sequence encoding these proteins to make the sequence compatible with human cells. In addition, PCT publication WO 99/49019 published Sep. 30, 1999 provides some sequence information regarding green fluorescent protein expressed from genes isolated from Renilla and Ptilocarpus. Gurskaya, N. G., et al., BMC Biochem (2002) 2:5 describes mutations which change the spectrum of emission of fluorescent protein from coral.

The above documents, each of which is incorporated herein by reference in its entirety, demonstrate that variations in the amino acid sequence of a protein which exhibits fluorescence upon excitation with radiation of shorter wavelength than the fluorescent wavelength provide a range of color choice and intensity. The fluorescent proteins have found wide use both in scientific research and in the production of novelty items, such as toys. Because the only requirements for fluorescence are irradiation with a suitable wavelength and because the fluorescence is visible to the naked eye, these proteins have proved convenient markers and have inspired whimsical applications.

It has now been found that additional variants of fluorescent protein have improved brightness and exhibit low toxicity. These proteins can also be modified to fluoresce in a variety of colors and to vary in intensity.

### DISCLOSURE OF THE INVENTION

The invention is directed to compositions and methods which employ fluorescent proteins that are related by homology to the protein encoded by the nucleotide sequence set forth in FIG. 1B. The exemplified GFP contains, in comparison to known fluorescent proteins, conservative substitutions and several substitutions which render it less acidic at the N-terminal portion but more acidic at the C-terminal portion. The invention is directed to compositions and methods related to a group of variants which are slightly less acidic in the N-terminal approximately half of the sequence and slightly more acidic at the C-terminal approximately half of the sequence. The proteins of the invention are non-toxic, and cells containing them can survive for at least 4 weeks and for 3, 4 or 6 months.

Thus, in one aspect, the invention is directed to the variant proteins per se and to methods to use the variants. In other aspects, the invention is directed to recombinant materials which encode the variants and methods to produce the variants using these materials, as well as other applications of the recombinant materials themselves.

### BRIEF DESCRIPTION OF THE DRAWINGS

FIG. 1A shows the deduced amino acid sequence of a variant of the invention designated herein A/C. FIG. 1B shows the nucleotide sequence of a nucleic acid, putatively isolated from R. reniformis, which encodes the amino acid sequence of FIG. 1A.

FIGS. 2A, 2B and 2C show nucleotide sequences based on the nucleotide sequence of FIG. 1B which are modified to conform to codon usage preferences for Saccharomyces cerevisiae, Escherichia coli and Bifidobacterium longum, respectively.

FIG. 3 shows a comparison of the amino acid sequence of the invention variant labeled A/C with a green fluorescent protein whose gene was cloned from R. mulleri.

FIGS. 4A and 4B show diagrams of a retroviral vector for production of the variants in various tumor cell lines. The vector is provided to packaging cells and the resulting virions used to infect tumor cell lines.

FIGS. 5A-5C show images of the fluorescent protein expressed in the packaging cell line PT67, the melanoma cell line B16F0, and the prostate cancer cell line PC3.

FIGS. 6A-6D show images of tumors grown from H46O, a lung cancer cell line, PC3, a prostate cancer cell line, CAPAN-1, a pancreatic cancer cell line and RKO, a colon cancer cell line.

FIGS. 7A and 7B show the nucleotide sequence encoding a fluorescent protein from coral, at positions 289-964.

### MODES OF CARRYING OUT THE INVENTION

Improved “green fluorescent proteins” (GFP) and recombinant materials which encode them are provided. This permits the use of a bright, nontoxic label to monitor gene expression, to label various cells, and to monitor the progress of metastases as described, for example, in PCT publication WO 98/49336, incorporated herein by reference. The improved fluorescent proteins of the invention offer more sensitive methods to assess these phenomena while remaining nontoxic to cells and entire organisms. Thus, the proteins of the present invention is useful in a variety of art known methods which employ the known forms of GFP described above. Production of GFP in general and use of recombinant materials as well as the GFP itself are well known in the art in view of the extensive literature describing the previously known forms of this fluorescent protein.

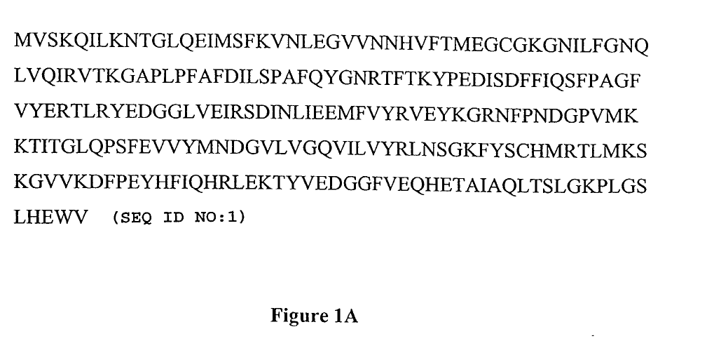

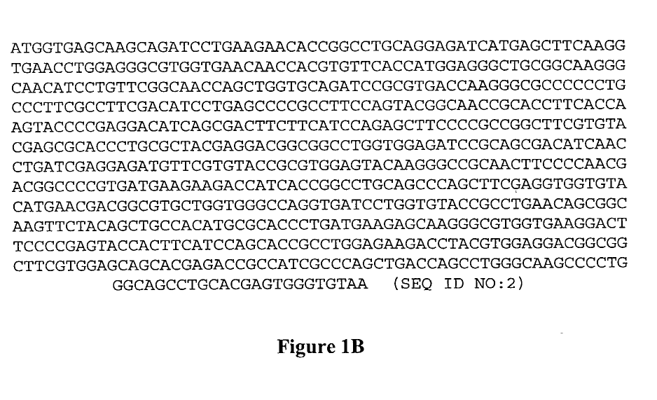

A protein of the amino acid sequence shown in FIG. 1A, designated A/C herein, emits green fluorescence and has the brightness and nontoxic properties stated above. However, as is known in the art, these “green” fluorescent proteins may be modified so that they fluoresce in various colors in the visible spectrum. Thus, by suitably modifying the amino acid sequence of the protein set forth in FIG. 1A, red, yellow, blue, or other color fluorescence may also be obtained. In addition, the brightness of the fluorescence can be varied by making small changes to the amino acid sequence. The nature of such modifications is helpfully described in, for example, U.S. Pat. No. 5,777,079 incorporated herein by reference above. As described, modifications to the serine residue which is found at position 66 of the A/C sequence can be replaced by alanine, leucine, cysteine, valine, isoleucine or threonine to obtain proteins with red shifted spectra which are generally brighter as compared to the unmodified form of A/C. Other modifications that appear to affect brightness or fluorescence wavelength include those at position 67. The chromophore appears to be focused on positions 66-68 of the A/C protein, which correspond to positions 65-67 of the Aequorea wildtype GFP protein discussed in the '079 patent. Thus, mutations at positions 66-68 are particularly important in modifying the properties of the protein.

Thus, the invention includes mutants having substitutions at any of positions 66-68 and particularly at position 66.

Additional modifications of 1-4 amino acids elsewhere in the molecule are also permitted; a minimal number of mutations of this type is insufficient to convert the A/C amino acid sequence into that of any known “green fluorescent protein” and has minimal impact on the properties of the molecule. Thus, the proteins of the invention include fluorescent proteins which are at least 90% homologous, preferably 95% homologous, and more preferably 99% homologous to the amino acid sequence shown in FIG. 1A. Particularly preferred are fluorescent proteins having the amino acid sequence shown in FIG. 1A and variants thereof which have at least one amino acid substitution in positions 66-68, preferably in position 66. Also preferred are modifications analogous to those described for the proteins from coral, described by Gurskaya, cited above.

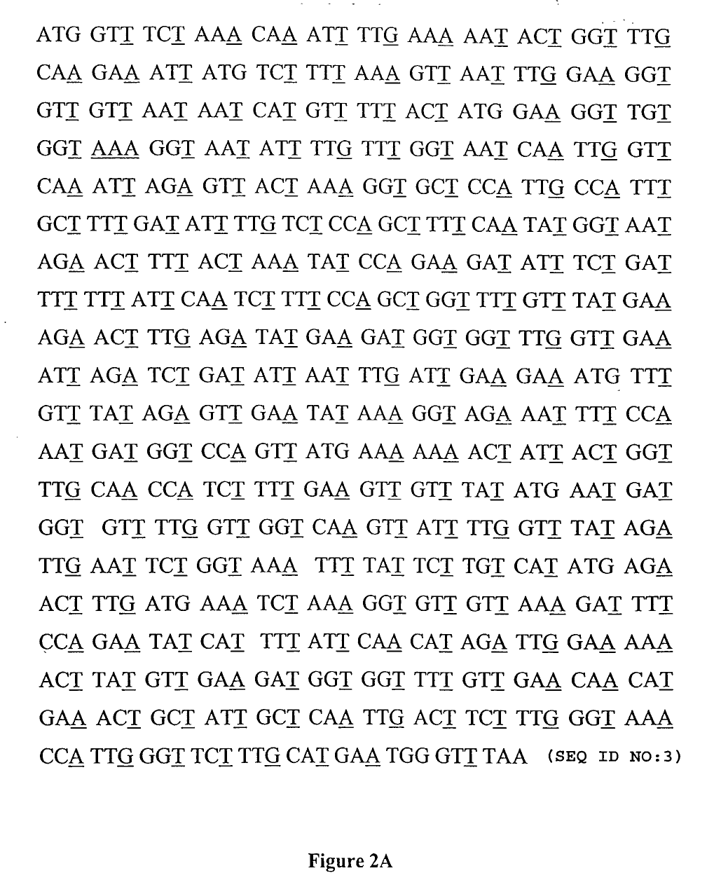

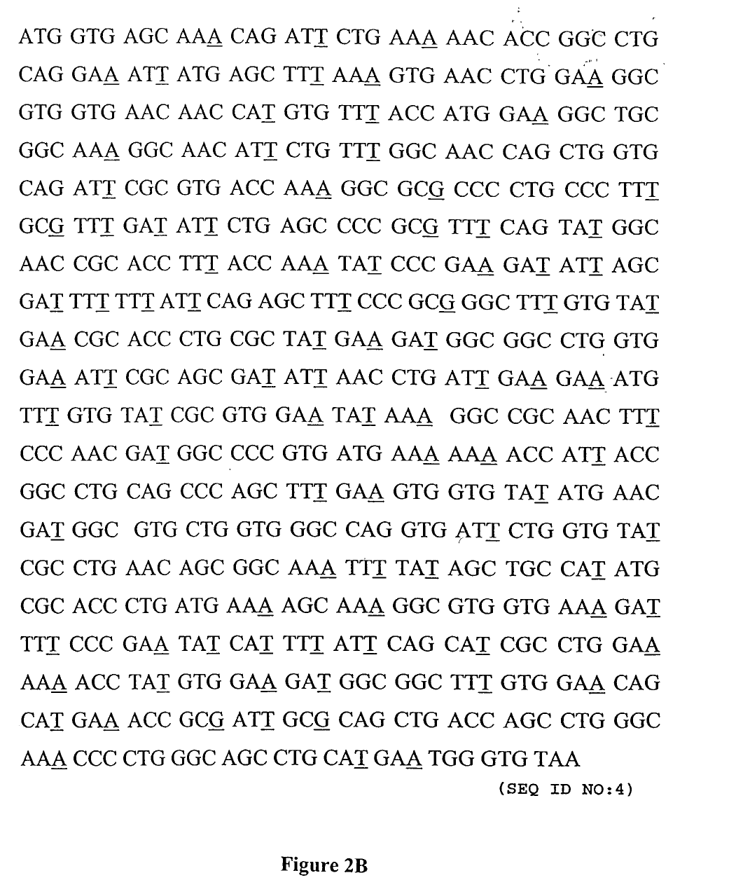

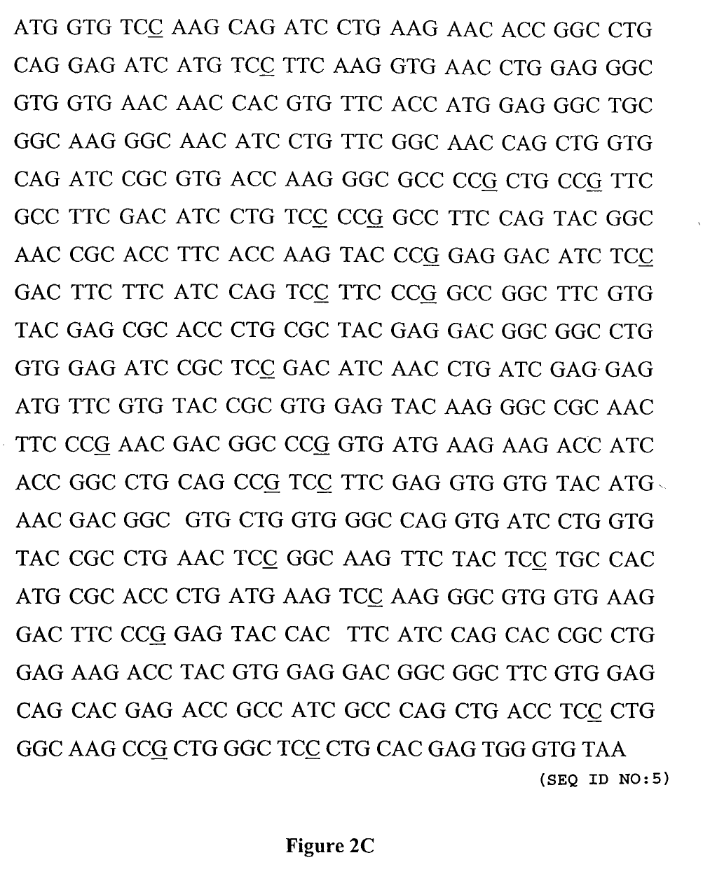

The nucleotide sequence encoding the fluorescent protein variants of the invention may be expressed in a wide variety of cells. Expression systems suitable for production of proteins from recombinant systems are by now conventional for prokaryotes, eukaryotes such as yeast and fungi, higher plants, animal cells, including vertebrate cells, mammalian cells, and especially human cells, and a variety of cell lines. The appropriate expression system and vector depends on the nature of the host and the application intended. In addition to providing an appropriate expression system, the nucleotide sequence may be modified to convert it to a preferred codon usage for the intended host. The nucleotide sequence shown in FIG. 1B, thus, may contain one or more of the modifications shown in FIGS. 2A, 2B and 2C for expression in the indicated hosts, Saccharomyces cerevisiae, Escherichia coli and Bifidobacterium longum, respectively. Thus, the sequence for expression in Saccharomyces cerevisiae may contain 1-230 silent base changes; that for E. coli from 1-94 silent base changes and that for Bifidobacterium longum 1-21 base changes. All intermediate numbers of base changes are also included within the scope of the invention.

The fluorescent proteins of the invention and the recombinant materials encoding them may be applied in a wide variety of uses as is set forth in detail in PCT publication WO 99/49019, cited above, and incorporated herein by reference. This publication describes a many uses, including analytical, research, diagnostic, and commercial uses.

Thus, the object of the production of the fluorescent proteins of the invention may be to obtain the protein itself for use in various compositions and articles of manufacture. These fluorescent proteins may be used in various items such as toys, dolls, card games, paints, textiles, balloons, cosmetics, and foodstuffs or any other article or composition designed to glow. The protein itself may be produced for incorporation into these articles and compositions. The fluorescent proteins of the invention may also be combined with other materials which fluoresce or emit light, such as luciferase. The '019 publication describes compositions in which other luminescent biological materials are combined in the same composition with fluorescent proteins so that rather than effecting excitation by irradiation from an external source, the irradiating wavelengths are generated in situ by the luminescent combined material.

More serious uses of the green fluorescent protein focus on its value as a research tool. In one embodiment, the fluorescent proteins of the invention may be fused or otherwise coupled to antibodies directed to target tissues in plants or animals. For example, it may be desirable to label tumors in animals and to follow metastases by coupling the fluorescent label to the tumor. The fluorescent proteins of the invention may be prepared as conjugates with moieties which are able to target tissues or cells. Typical targeting moieties are specific binding partners for a material displayed on tissues or cells. Typical targeting moieties are antibodies and ligands for receptors.

Alternatively, the production of a fusion protein containing a green fluorescent protein can be used to monitor expression of the coupled protein. In addition, as described, for example, by Yang, M, et al., Cancer Res. (1998) 58:4217-4221 and by Yang, M, et al., Cancer Res. (1999) 59:731-736 and as reviewed by Hoffman, R. M., Cancer & Metastasis Reviews (1999) 17:271-277, fluorescent proteins may be generated in tumors and used to monitor metastasis.

The fluorescent proteins of the invention may also be used to label reagents in assays such as immunoassays. In one example, a sandwich assay may be employed wherein one specific binding partner to an analyte is a capture moiety which immobilizes the analyte and a second specific binding partner is used to label the immobilized analyte. The fluorescent proteins of the invention may be used directly as a label on the labeling binding partner or on a secondary binding partner such as, for example, the use of a second antibody-bearing label to couple to a first antibody directly bound to analyte.

Expression of a protein can also be monitored by fusing the nucleotide sequence encoding the protein to a nucleotide sequence encoding the fluorescent protein of the invention. Expression of the protein of interest may then be determined by monitoring the fluorescence generated as the fusion protein is produced. Alternatively, the capacity of a promoter or other control sequence to effect expression can be monitored by placing a nucleotide sequence encoding the invention fluorescent protein in operable linkage therewith. Again, fluorescence is generated by virtue of production of the fluorescent protein, thereby indicating that the expression controls are operable.

The foregoing applications are merely exemplary. In general, the fluorescent proteins of the invention can be used in any application where fluorescent labels are employed, including assay modifications such as fluorescence polarization and fluorescence quenching assays. The fluorescent proteins of the invention have the additional advantage of the capability of being generated in situ so that their presence or absence or amount can be used as an index of expression as well as to provide an internal source of fluorescence in cells. Thus, the course of tumor metastasis, bacterial or viral infection, or the movement of cells of any type within a plant or animal organism can be traced using the fluorescent proteins of the invention.

The following example is intended to illustrate but not to limit the invention.

### EXAMPLE 1

### Expression of Improved Fluorescent Protein

A nucleic acid molecule encoding the fluorescent protein having the amino acid sequence shown as A/C in FIG. 1A was obtained, labeled R. reniformis GFP, from Stratagene (San Diego, Calif.). The nucleotide sequence encoding the protein was determined and is shown in FIG. 1B.

The nucleotide sequence is optionally modified as shown in FIGS. 2A-2C by silent base substitutions so as to optimize expression in various host organisms.

The amino acid sequence of the fluorescent protein, in comparison to the fluorescent protein in the art to which applicants believe it is most closely related, is shown in FIG. 3. The A/C protein is approximately 86% identical to the amino acid sequence of a protein encoded by a nucleotide sequence isolated from R. mulleri. The chromophore portions of these proteins, positions 66-68 of A/C and positions 65-67 of R. mulleri, are identical.

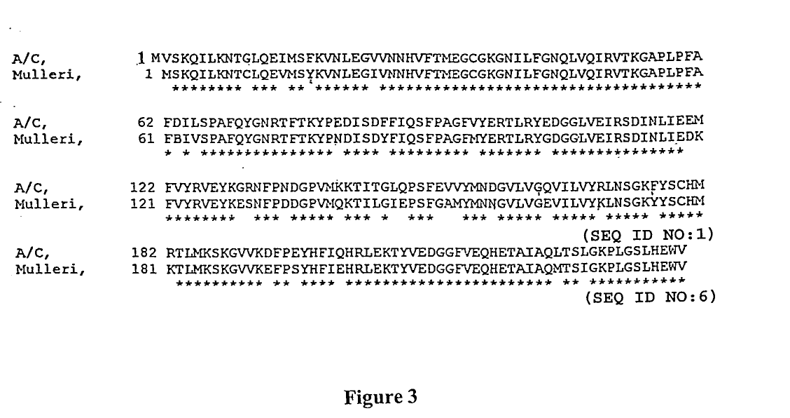

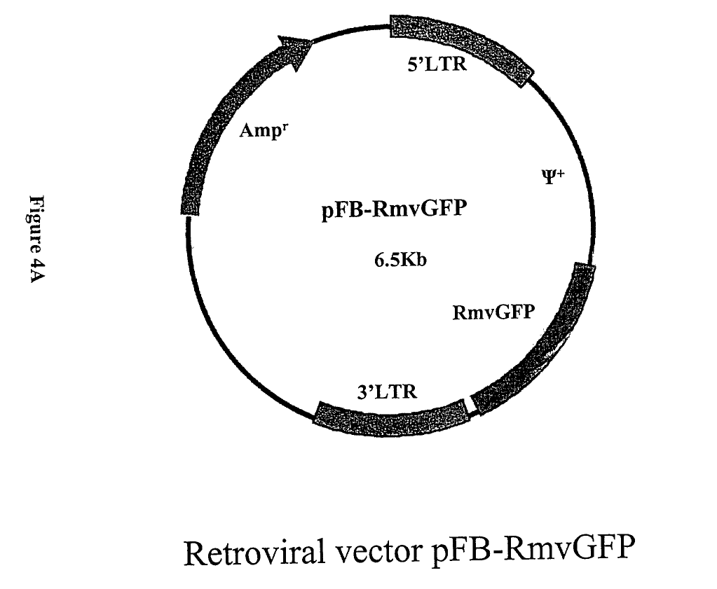

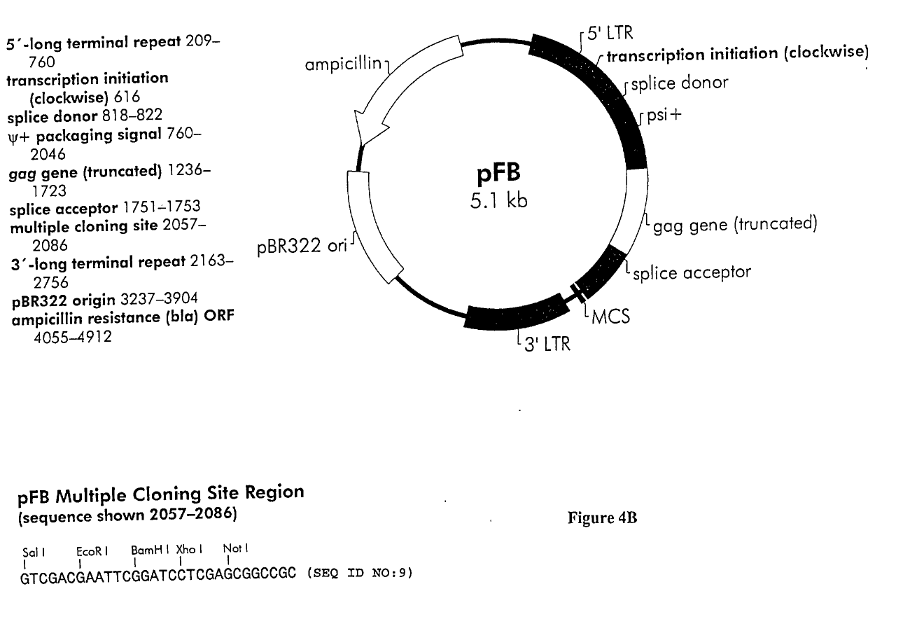

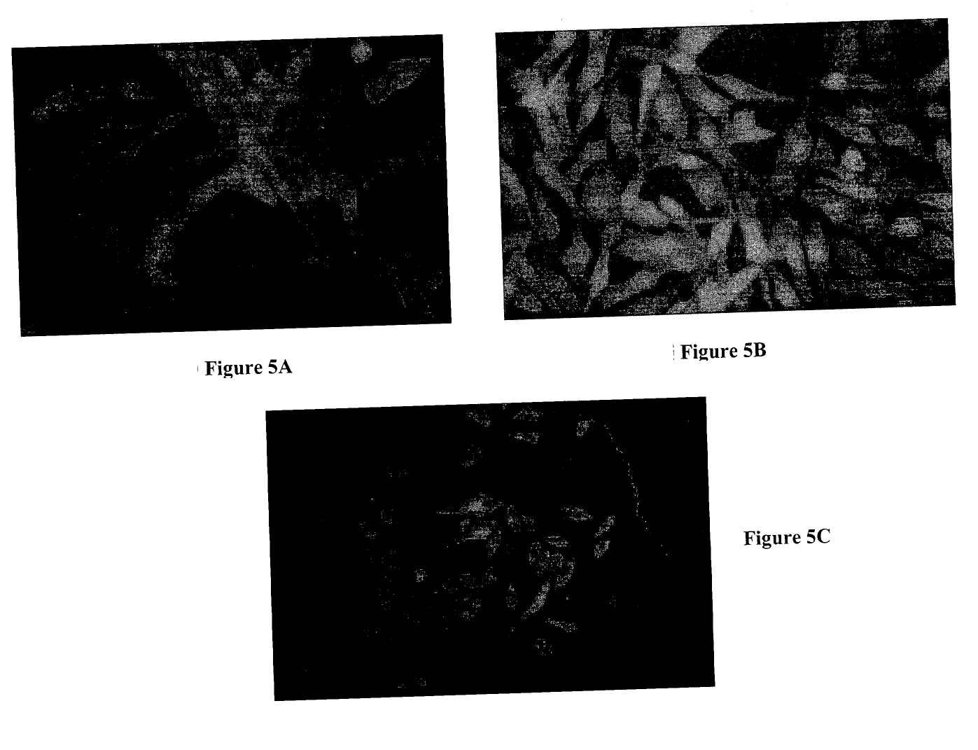

The nucleotide sequence of FIG. 1B is inserted into a retroviral vector under control of a 3′ LTR promoter (FIG. 4A). In this vector, derived from pFB (Stratagene) shown in FIG. 4B, the multiple cloning site region is shown. The resultant vector, designated pFB-Rmv GFP, was used to modify PT-67 packaging cells. The packaging cells were cultured, producing a bright green signal due to the production of the GFP protein. See FIG. 5A. High intensity was maintained for over 10 days, indicating the GFP is nontoxic.

### EXAMPLE 2

### Preparation of Cell Lines Expressing A/C Fluorescent Protein

A number of tumor cell lines were infected with the packaged virions prepared in the PT-67 packing cell line and are as follows: The breast cancer cell line MX-1; the prostate cancer cell lines MDA-PCA2B and PC3; the pancreatic cancer cell line CAPAN-1; the colon cancer cell line RKO; the sarcoma cell line MES-SA/DX5; the lung cancer cell lines H460, Lewis lung, and A549 and the melanoma line B16F0. Several of these are illustrated. The fluorescence of these cell lines is illustrated in FIGS. 5B (B16F0) and 5C (PC3). The fluorescence is maintained for weeks.

### EXAMPLE 3

### Tumor Labeling

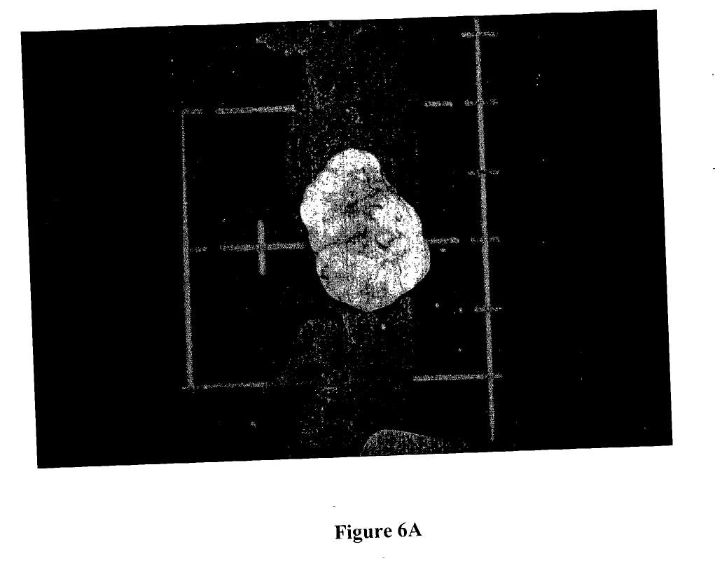

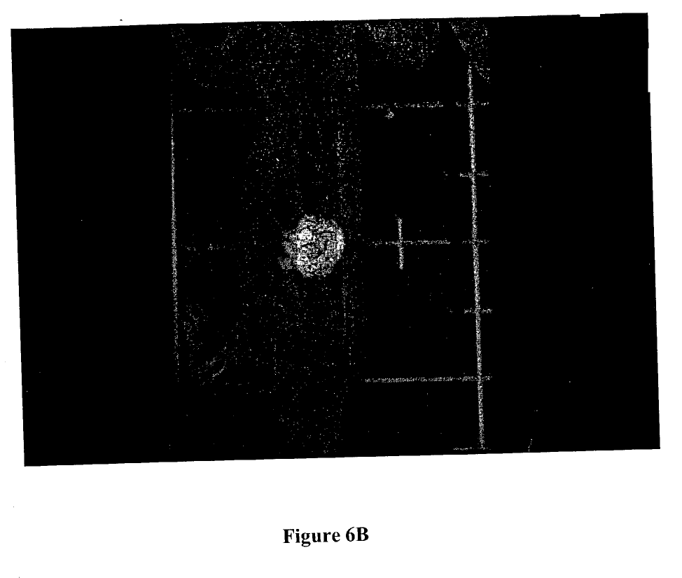

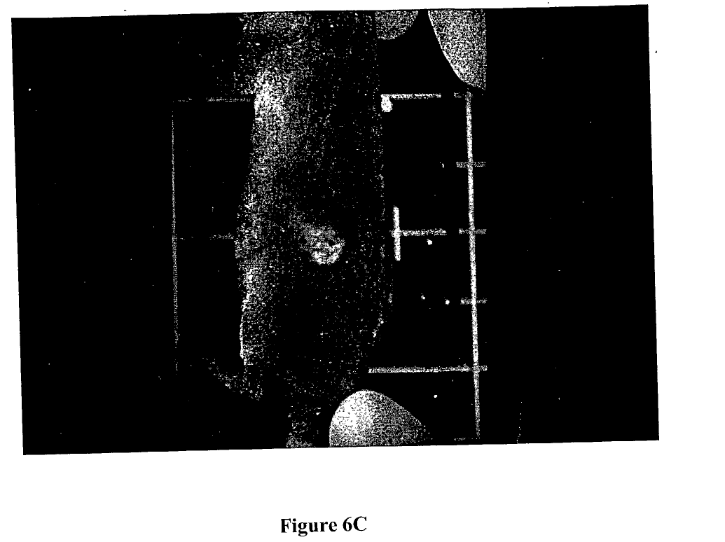

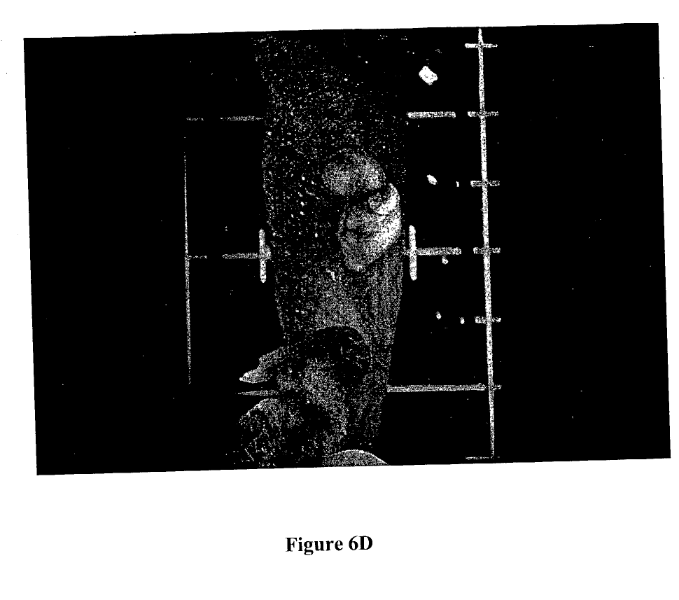

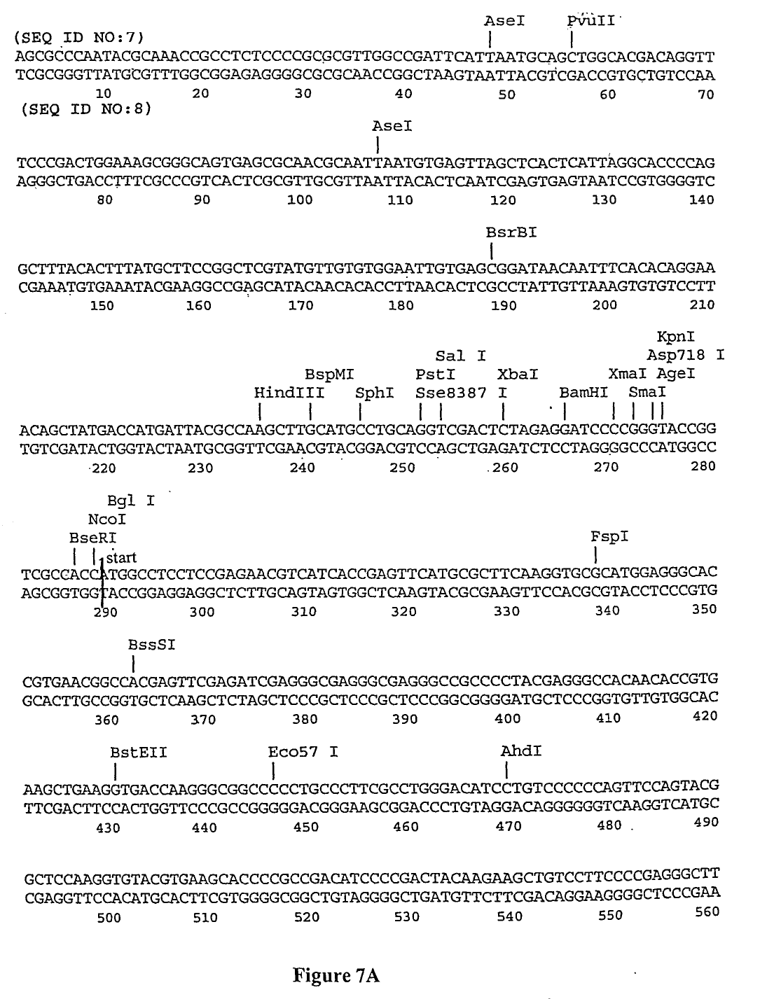

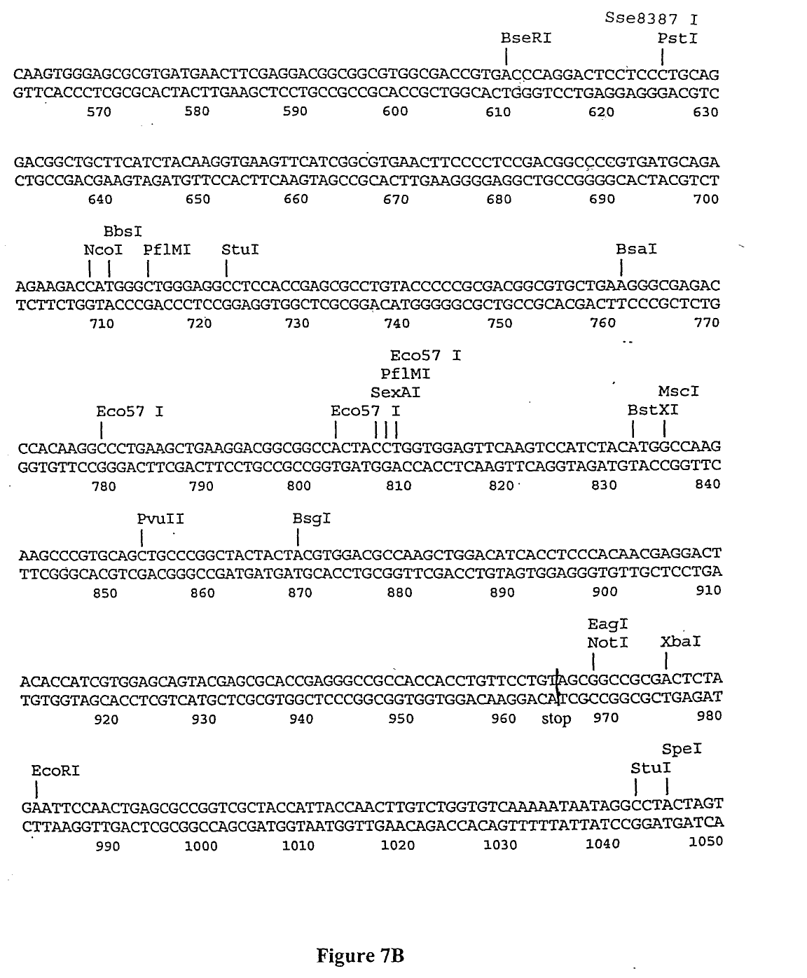

Tumors were grown from the transformed cell lines by subcutaneous injection in immunocompromised mice and the progress of the tumor followed by monitoring fluorescence. Illustrative results are shown in FIGS. 6A-6D.

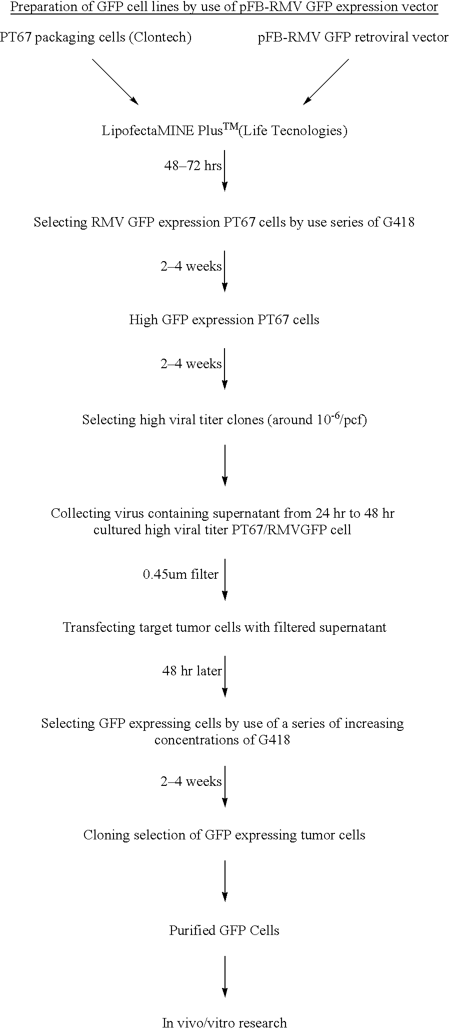

1. A fluorescent protein comprising the amino acid sequence of the A/C protein of FIG. 1A or a fluorescent protein having at least 90% homology with said A/C protein.

2. The fluorescent protein of claim 1, which has at least 95% homology with the A/C protein of FIG. 1A.

3. The fluorescent protein of claim 2, which has at least 99% homology with the A/C protein of FIG. 1A.

4. The fluorescent protein of claim 1, which comprises the amino acid sequence of the A/C protein of FIG. 1A.

5. The fluorescent protein of claim 1, wherein at least 1 amino acid in positions 66-68 has been replaced by a different amino acid.

6. The fluorescent protein of claim 5, wherein the amino acid at position 66 has been replaced.

7. A nucleic acid molecule comprising a nucleotide sequence which encodes the fluorescent protein of claim 1.

8. A nucleic acid molecule comprising a nucleotide sequence which encodes the fluorescent protein of claim 4.

9. The nucleic acid molecule of claim 8, which comprises the nucleotide sequence set forth in FIG. 1B, optionally containing 1 or more nucleotide substitutions as set forth in FIGS. 2A, 2B or 2C.

10. A recombinant expression system which comprises a nucleotide sequence encoding the fluorescent protein of claim 1 operably linked to control sequences for its expression.

11. A recombinant host cell which comprises the expression system of claim 10.

12. A method to produce a fluorescent protein, which method comprises culturing the cells of claim 11, wherein said nucleotide sequence is expressed to produce said fluorescent protein, and optionally recovering said fluorescent protein.

13. Antibodies specifically immunoreactive with the fluorescent protein of claim 1.

14. A conjugate comprising the protein of claim 1 coupled to a targeting moiety.

15. The conjugate of claim 14, wherein said targeting moiety is an antibody or a ligand for a receptor.

16. The expression system of claim 10, wherein said nucleotide sequence encoding the fluorescent protein is fused to a nucleotide sequence encoding additional protein.

17. A method to monitor the production of a protein, which method comprises
providing an expression system which comprises a first nucleotide sequence encoding said protein fused to a second nucleotide sequence encoding the fluorescent protein of claim 1;
placing said expression system in an environment in which expression is to be monitored; and
assessing generation of fluorescence, whereby generation of fluorescence indicates expression of said protein.

18. A method to evaluate the activity of a promoter, which method comprises providing a nucleic acid which comprises said promoter operably linked to a nucleotide sequence encoding fluorescent protein of claim 1;
placing said nucleic acid in an environment in which the activity of the promoter is to be evaluated;
monitoring appearance and amount of fluorescence;
wherein the appearance and amount of fluorescence indicates the activity of the promoter.

19. A method to label tissue, which method comprises contacting said tissue with the conjugate of claim 14, wherein said targeting moiety is specific for said tissue.

20. A method to assess the position and progression of tumors and their metastases, which method comprises modifying said tumors to express the fluorescent protein of claim 1 and observing the position of fluorescence.

21. An improved method to conduct an immunoassay, wherein said immunoassay comprises entrapping analyte or an analog thereof in a sandwich comprising a first specific binding partner for said analyte coupled to a solid support and a second specific binding partner for said analyte comprising a label wherein the improvement comprises employing as a label the fluorescent protein of claim 1.
